(19) United States
(12) Patent Application Publication
(10) Pub. No.: US 2009/0197974 A1
(43) Pub. Date: Aug. 6, 2009

(54) NATURAL DIETARY SUPPLEMENT TABLET

(76) Inventors: Khaleeq Ahmed, Woodstock, IL (US); Douglas J. Basten, Green Bay, WI (US); Scott P. Brady, Seymour, WI (US); Robert C. Doster, Oneida, WI (US); Lisa A. Evenson, Manitowoc, WI (US); Meganne W. Finerty, Appleton, WI (US); Dan J. Hnilicka, Depere, WI (US); Janelle T. Jandrain, Green Bay, WI (US); Richard L. Sharpee, Green Bay, WI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/168,521

(22) Filed: Jul. 7, 2008

Related U.S. Application Data
(60) Provisional application No. 61/026,301, filed on Feb. 5, 2008.

(51) Int. Cl. A61K 47/36

(52) U.S. Cl. 514/777

(57) ABSTRACT

An all-natural or substantially all-natural dietary supplement in a tablet form that comprises at least 95 percent Certified Organic ingredients by weight and exhibits desirable hardness, friability and release of the active dietary supplement is formulated with naturally occurring saccharides that exhibit tablet binder, tablet disintegrant, or both tablet binder and tablet disintegrant functionality. The resulting tablets may be labeled as “natural” dietary supplements.

### CROSS REFERENCE TO RELATED APPLICATIONS

This application claims, under 35 U.S.C. §119(e), the benefit of U.S. Provisional Application No. 61/026,301, filed Feb. 5, 2008, entitled “NATURAL DIETARY SUPPLEMENT TABLET,” the entire disclosure of which is incorporated herein by reference.

### FIELD OF THE INVENTION

This invention relates to the field of nutritional supplementation, and more particularly to solid orally administered dietary supplements in a tablet form.

### BACKGROUND OF THE INVENTION

A variety of dietary supplements have long been available in tablet form, including natural dietary supplements. More recently, dietary supplements comprised of Certified Organic ingredients have been marketed. However, known dietary supplement tablets have generally employed conventional binders, fillers, disintegrants, lubricants, and/or other excipients that either are not or cannot be certified as organic ingredients pursuant to the requirements of the Organic Foods Production Act of 1990 (7 U.S.C. §6501 et seq.).

Other attempts to satisfy the demand products containing natural dietary supplements have generally involved preparation of liquid or gel filled capsules, especially soft gelatin capsules. However, these capsules are not Certified Organic since the raw materials used to make the capsules are not organic or organic compliant, and do not meet the 95% Certified Organic requirement.

In terms of consumer preference, as it relates to pharmaceutical dosage forms, hard gelatin capsules are generally considered less desirable than either tablets or soft gelatin capsules. Although there are slightly more consumers having a preference for soft gelatin capsules as opposed to tablets, it is believed that this slight preference is attributable to faster release and absorption of the active ingredient, whereby faster relief from the symptoms treated by the active is achieved. It is also believed that the slight preference for soft gelatin capsule pharmaceutical dosage forms could be attributable to a historically based perception that soft gelatin capsules may be more potent than tablets. It is believed that such considerations are not relevant to dietary supplements, and that dietary supplements in tablet form will be preferred by many consumers. Further, tablet forms have many advantages over soft gelatin capsule forms, including lower manufacturing costs, reduced susceptibility to tampering, and reduced issues with stability, discoloration and development of turbidity. Further, tablets are not susceptible to leakage as are soft gelatin capsules.

However, it is believed that attempts at preparing all-natural or substantially all-natural dietary supplements in a tablet form comprised entirely or almost entirely of Certified Organic ingredients have yielded unacceptable results. Specifically, it is believed that such attempts have not resulted in tablets having a suitable hardness, friability and/or release profile of the active, and/or have involved the use of tablet formulations having undesirable processing characteristics, such as poor flow properties, poor compressibility and/or a tendency to stick to the dies during tablet compression.

To the knowledge of the inventor, there have not been any reported or commercially available dietary supplements in tablet forms that are comprised entirely or almost entirely of Certified Organic ingredients prior to this invention. Accordingly, there is a need for improved dietary supplement tablets comprised entirely or almost entirely of Certified Organic ingredients, and processes for preparing such tablets.

### SUMMARY OF THE INVENTION

In an aspect of the present invention there is provided an all-natural or substantially all-natural dietary supplement in a tablet form, wherein the tablet form comprises at least 95% Certified Organic ingredients by weight. Such all-natural dietary supplement tablet includes at least one Certified Organic dietary supplement and at least a first tablet excipient selected from naturally occurring substances, wherein the first tablet excipient is present in an amount effective to act as a tablet binder, a tablet disintegrant, or both a tablet binder and a tablet disintegrant.

In accordance with certain aspects of the invention, the dietary supplement in a compressed tablet form comprises from 5% to 80% by weight of a combination of at least two monosaccharides, disaccharides, oligosaccharides, or polysaccharides, wherein at least one of the tablet excipients is a dietary fiber, and wherein the combination of tablet excipients act as tablet binders, tablet disintegrants, or both tablet binders and tablet disintegrants.

In accordance with certain aspects of the invention, there is provided a process for making a dietary supplement in a tablet form, wherein the resulting tablet contains at least 95% Certified Organic ingredients by weight. The process involves steps of providing a selected Certified Organic dietary supplement in a nutritionally effective amount; optionally granulating the dietary supplement with a Certified Organic ingredient that acts as a granulating agent; providing at least one tablet excipient selected from naturally occurring substances in an amount effective to act as a tablet binder, a tablet disintegrant, or both a tablet binder and a tablet disintegrant; blending the dietary supplement or granulated dietary supplement with the tablet excipient or a combination of tablet excipients and optionally with other ingredients to provide a tablet formulation; and compressing the tablet formulation to form a tablet.

These and other features, advantages and objects of the present invention will be further understood and appreciated by those skilled in the art by reference to the following specification and claims.

### DETAILED DESCRIPTION OF PREFERRED EMBODIMENTS

It has been discovered that certain Certified Organic ingredients may be employed in nutritional tablet dosage forms in place of conventional synthetic or other excipients that are not organic compliant to provide a tablet formulation having good processability characteristics and which can be compressed to form tablets having desirable hardness, friability and release properties. As a result of these discoveries, all-natural or substantially all-natural dietary supplements comprising at least 95% Certified Organic ingredients can be prepared.

As used herein, the term “Certified Organic ingredients” means ingredients that are certified “Organic” by the U.S.D.A. Accredited Certifying Agents for the National Organic Program established in accordance with the Organic Foods Production Act of 1990 (7 U.S.C. 6501 et seq.). Certified Organic ingredients are also compliant with processing standards for growing, storing, processing, packaging and shipping to avoid synthetic chemicals, genetically modified organisms, irradiated materials and sewage sludge.

Generally, “Certified Organic” ingredients or products are those that do not contain any artificial flavoring, coloring or chemical preservative (as defined by 21 CFR 01.22) or any other artificial or synthetic ingredient. Additionally, Certified Organic ingredients or products generally are not subjected to processing or are only subjected to minimal processing. Minimal processing may include traditional processes used to make a product edible or to preserve it or to make it safe for human consumption (e.g., smoking, roasting, freezing, drying or fermenting), or other physical processes which do not fundamentally alter the raw product and/or which only separate a whole intact food into component parts (e.g., grinding, separating, pressing).

Excluded from the category of “Certified Organic products” are most of the commonly employed tablet excipients including microcrystalline cellulose, cellulose derivatives, starch derivatives, polyvinylpyrrolidone, and other synthetic or highly processed lubricants and disintegrants.

The inventors have discovered that a limited number of Certified Organic ingredients can function as binders, disintegrants and hardness regulators. The Certified Organic ingredients that may be employed as excipients in the tablets and methods of the invention are generally naturally occurring substances, although they may be concentrated or purified from processes that do not alter their fundamental characteristics and do not prevent them from being Certified Organic products or ingredients. Among the Certified Organic ingredients, dextrose, inulin, soy fiber, oat fiber, guar gum, gum Arabic, locust bean gum, gum acacia were identified as suitable excipients for tableting. Certified Organic ingredients that have been identified as having tablet binder and/or tablet disintegrating properties may be categorized as saccharides, including monosaccharides, disaccharides, oligosaccharides, and polysaccharides, the polysaccharides including dietary fiber. While there is some overlap in functionality (some of the identified excipients exhibit both tablet binding and tablet disintegration properties), and some observed synergy among combinations, certain dietary fibers, such as soy fiber and/or oat fiber may be used effectively as tablet disintegrants, optionally in combination with effervescent combinations. Inulin (a soluble dietary fiber) and dextrose have been found to provide excellent tablet binding properties when used in the invention. Inulin also may be used to enhance flowability of a powder formulation.

In addition to their use as disintegrants, dietary fibers, such as soy fiber and oat fiber, may be suitably employed as diluents, and are capable of providing some binder functionality, especially when employed in combination with other saccharides such as inulin and/or dextrose.

In general, it has been found that the identified Certified Organic disintegrants, optional effervescing auxiliary disintegrants, and/or binders may be selected and employed in suitable amounts to adjust tablet hardness, friability and release of the active agent or dietary supplement. For example, softer tablets may be achieved by using lower amounts of binder and disintegrant. Harder tablets may be achieved by increasing the amount of binder and/or granulating the active ingredient (e.g., dietary supplement) and/or other ingredients. Similarly, friability may be modified by appropriate adjustment of the amount of binder, disintegrant and/or granulation. Generally, tablet disintegration and the rate at which the active ingredient (e.g., Certified Organic dietary supplement) is released can be increased by adding more disintegrant or a second disintegrant, such as an effervescent combination. Suitable effervescent combinations may comprise an effervescing carbonate and/or bicarbonate salts such as sodium bicarbonate, potassium bicarbonate and/or calcium carbonate, and an organic compliant acid such as citric acid.

In some cases, it may be necessary and/or desirable to incorporate a tablet lubricant into the formulation to prevent the formulation from sticking to the punches and dies of a tableting machine. Conventional tablet lubricants, such as stearic acid and its salts, are neither Certified Organic ingredients nor organic compliant materials. However, in accordance with certain aspects of the invention, it has been discovered that a Certified Organic oil plated on surfaces of a Certified Organic polysaccharide, such as maltodextrin or gum acacia, may be employed in amounts of from about 0.1 percent by weight to about 3 percent by weight as a tablet lubricant to provide a tablet formulation in accordance with the invention that resists sticking to the dies and punches of a tableting machine. Suitable Certified Organic oils include properly certified vegetable oils, such as safflower oil, canola oil, palm oil and the like. In general, the lubricants may be prepared by mixing or blending the Certified Organic oil with the polysaccharide. The amounts and mixing/blending times are selected to distribute the oil on surfaces of the polysaccharide. Suitable proportions include from about 20 percent to about 40 percent oil and from about 80 percent to about 60 percent polysaccharide.

As previously stated, oil plated onto maltodextrin may be used to help lubricate. Lubrication is used to prevent sticking of the product to the punches and dies allowing good release. However, there are other benefits for lubrication, which help the appearance of the product and the disintegration of the tablet so that the active is available for the body to use.

The dietary supplement tablets of this invention preferably include at least 95 percent Certified Organic ingredients by weight, the remaining ingredients comprising non-agricultural substances allowed as ingredients in or on products labeled as “Organic” as specified by 7 U.S.C. §205.605, and may comprise as much as 100 percent Certified Organic ingredients. Organic compliant, or non-agricultural substances (as listed under 7 U.S.C. §205.605) that may be employed in the tablets according to the invention include small amounts of organic compliant acids (e.g., citric acid and/or lactic acid) in combination with an effervescing carbonate and/or bicarbonate salt (e.g., sodium bicarbonate) to supplement disintegration with an effervescent combination or system, silicon dioxide as a flow or processability aid, etc. In general, effective amounts of flow aids and effervescent combinations, if employed at all, comprise much less than 5 percent by weight of the tablet, whereby the tablet may be labeled “Organic.” Whenever additional flowability is an issue, silicon dioxide can be combined with the Certified Organic dietary supplement prior to adding the disintegrant and binder. Whenever binding is a problem, adding gum arabic or acacia gum can be used for either wet or dry granulation prior to adding the disintegrant and binder.

The term “saccharide” as used herein refers to monosaccharides, disaccharides, oligosaccharides and polysaccharides. The expression “oligosaccharide” as used herein refers to non-polymeric compounds having three or more monosaccharide units linked together. Polysaccharides have a sufficiently large number of repeat units such that the addition of another repeat unit to the polymer chain does not appreciably change the chemical and/or physical properties of the molecule. For purposes of this disclosure, the expression “oligosaccharide” is meant to encompass compounds having up to about 10 repeat units, and the expression “polysaccharide” is meant to encompass compounds having more than about 10 repeat units.

The expression “dietary fiber” as used herein refers to a polysaccharide that is resistant to digestion and absorption in the human small intestine, but which may undergo complete or partial fermentation in the large intestine and/or colon.

Active dietary ingredients or dietary supplements that may be formulated with the Certified Organic excipients and compressed into tablets in accordance with this invention include those materials identified or defined as dietary supplements by 21 U.S.C. §321. In general, such active ingredients include various nutrients including vitamins, minerals, herbs, or other botanicals, amino acids, and dietary substances used to supplement the diet by increasing the total dietary intake. Also included are concentrates, metabolites, constituents, extracts and combinations thereof of the above-referenced vitamins, minerals, herbs, amino acids, and substances for increasing the total dietary intake.

In particular examples, organic soy fiber and/or organic oat fiber used in an amount of from about 5 percent by weight to about 50 percent by weight of a tablet formulation acted as diluents and disintegrants in organic tablets in which the active dietary supplement was green tea, garlic or grape seed. Specifically, soy fiber was employed in an amount of about 21 percent by weight in green tea tablets, oat fibers were employed in an amount of about 16 percent in garlic tablets, and a combination of about 14 percent oat fiber and about 20 percent soy fiber was employed in a grape seed tablet.

Also in accordance with these examples, organic inulin (commonly derived from chicory, yucca or Jerusalem artichoke) was employed as a binder in the green tea tablet, the garlic tablet, and the grape seed tablet. Suitable binding properties were achieved with 41.5 percent in the green tea tablet, about 22 percent in the garlic tablet, and about 20 percent in the grape seed tablet. Over 50 percent Organic inulin was used in a gingko and milk thistle tablet. Granulation may be employed to reduce the amount of inulin needed for binding of either sticky fruit and root powders or oily botanical seed powders.

In general, the choice and amount of binder, diluent, disintegrant and/or lubricant may be selected so that the tablets can be made for a large variety of nutritionally active organic ingredients, with the only other changes involving granulation to control particle size, and/or the addition of an agent to control flow. Because of the relatively high amount of variability from lot to lot with natural and organic components, granulation (using deionized water) may be employed to improve the consistency of the particle size from lot to lot and allow more consistent processing and final product from lot to lot.

Properties of the active ingredients that are important in making an appropriate selection include the percent oil, the percent sugars, the percent moisture, the particle size and particle size distribution, hygroscopicity and the amount of active needed in a unit dose. In general, oat fiber is preferred for active materials having a high oil content and soy fiber for active materials that are in a sticky powder form. Since it has higher fat content and lubricates sticky materials, as a general rule, oat fiber has better oil absorption properties, but has more gumminess, so it may be stickier in the presence of sugars. Disintegration, dissolution, friability and hardness are all tests that indicate quality and success for tableting. Dissolution testing is generally used for ensuring release of the individual active compound or compounds in an ingredient. Chewable tablets may have different friability and hardness criteria than swallowable tablets. Dissolution is not usually a concern with chewable tablets. Typically, soy fiber and/or oat fiber may be added as diluents in an amount that imparts good flow properties. The lubricant in accordance with the invention is preferably added in an amount that is the lowest level necessary to prevent sticking of the formulation in the die and punches of a tableting machine. A selected Certified Organic binder is added in an amount that is effective to form a tablet in a tableting machine. Once a tablet is made, optimization can be achieved based on disintegration and friability results.

Examples of formulations that either have or are expected to be successfully produced are listed in the following tables.

ACEROLA

| Tablet Core Ingredients | Mass/Tablet | |
|---|---|---|
| Organic Acerola (spray dried powder) | 500.0 | mg |
| Organic Soy Fiber | 100.0 | mg |
| Organic Inulin | 200.0 | mg |
| Sodium Bicarbonate (USP) | 7.0 | mg |
| Citric Acid (USP fine) | 6.0 | mg |
| Organic Safflower Oil | 40.0 | mg |
| Silicon Dioxide | 15.0 | mg |

CHERRY FRUIT

| | Mass/Tablet | |
|---|---|---|
| Tablet Core Ingredients | | |
| Sodium Bicarbonate (USP) | 7 | mg |
| Citric Acid (USP fine) | 7 | mg |
| Silicon Dioxide | 7 | mg |
| Organic Cherry Fruit (10:1) | 500 | mg |
| Organic Oat Fiber | 250 | mg |
| Tablet Coating Ingredients | | |
| Carageenan | 7.00 | mg |
| Glycerin USP Nat. | 0.71 | mg |

ALTERNATIVE CHERRY FRUIT

| | Mass/Tablet | |
|---|---|---|
| Tablet Core Ingredients | | |
| Organic Soy Fiber | 500.0 | mg |
| Sodium Bicarbonate USP | 11.0 | mg |
| Citric Acid (USP fine) | 10.0 | mg |
| Silicon Dioxide | 15.0 | mg |
| Organic Cherry Fruit 10:1 | 500.0 | mg |
| Tablet Coating Ingredients | | |
| Carageenan | 8.00 | mg |
| Glycerin USP Nat. | 0.75 | mg |

CHEWABLE ACEROLA

| Tablet Core Ingredients | Mass/Tablet | |
|---|---|---|
| Organic Acerola (spray dried powder) | 750.0 | mg |
| Organic Inulin | 600.0 | mg |
| Citric Acid (USP fine) | 11.0 | mg |
| Organic Safflower Oil | 130.0 | mg |
| Silicon Dioxide | 25.0 | mg |
| Organic Cherry Fruit (10:1) | 60.0 | mg |
| Natural Sour Cherry Flavor | 40.0 | mg |
| Organic Tapioca Dextrose | 600.0 | mg |

KELP

| | Mass/Tablet | |
|---|---|---|
| Tablet Core Ingredients | | |
| Organic Inulin | 410.0 | mg |
| Sodium Bicarbonate (USP) | 17.0 | mg |
| Citric Acid (USP fine) | 13.0 | mg |
| Organic Safflower Oil | 10.0 | mg |
| Organic Kelp | 250.0 | mg |
| Tablet Coating Ingredients | | |
| Carageenan | 3.33 | mg |
| Glycerin USP Nat. | 0.18 | mg |

BILBERRY

| | Mass/Tablet | |
|---|---|---|
| Tablet Core Ingredients | | |
| Organic Soy Fiber | 75.0 | mg |
| Organic Inulin | 170.0 | mg |
| Sodium Bicarbonate (USP) | 7.0 | mg |
| Citric Acid (USP fine) | 4.0 | mg |
| Organic Safflower Oil | 5.0 | mg |
| Silicon Dioxide | 4.0 | mg |
| Organic Oat Fiber | 75.0 | mg |
| Organic Bilberry (Non-granulated) | 60.0 | mg |
| Tablet Coating Ingredients | | |
| Carageenan | 3.800 | mg |
| Glycerin USP Nat. | 0.200 | mg |

GRAPE SEED

| | Mass/Tablet | |
|---|---|---|
| Tablet Core Ingredients | | |
| Organic Soy Fiber | 100.0 | mg |
| Organic Inulin | 100.0 | mg |
| Sodium Bicarbonate (USP) | 10.0 | mg |
| Citric Acid (USP fine) | 5.0 | mg |
| Organic Safflower Oil | 11.0 | mg |
| Silicon Dioxide | 5.0 | mg |
| Organic Oat Fiber | 70.0 | mg |
| Organic Grape Seed Extract (60%) | 200.0 | mg |
| Tablet Coating Ingredients | | |
| Carageenan | 4.76 | mg |
| Glycerin USP Nat. | 0.251 | mg |

GREEN TEA

| | Mass/Tablet | |
|---|---|---|
| Tablet Core Ingredients | | |
| Organic Soy Fiber | 260.0 | mg |
| Organic Inulin | 200.0 | mg |
| Sodium Bicarbonate (USP) | 10.0 | mg |
| Citric Acid (USP fine) | 6.0 | mg |
| Organic Safflower Oil | 80.0 | mg |
| Silicon Dioxide | 20.0 | mg |
| Organic Green Tea | 315.00 | mg |
| Tablet Coating Ingredients | | |
| Carageenan | 8.22 | mg |
| Glycerin USP Nat. | 0.69 | mg |

ORGANIC LUTEIN

| Tablet Core Ingredients | Mass/Tablet | |
|---|---|---|
| Organic Inulin | 140.0 | mg |
| Organic Safflower Oil | 23.0 | mg |
| Organic Spinach Powder | 1,000.00 | mg |
| (with 700 PPM Lutein) | | |
| Organic Gum Arabic | 36.00 | mg |
| Ferrous Succinate | 48.65 | mg |

MILK THISTLE

| Tablet Core Ingredients | Mass/Tablet | |
|---|---|---|
| Organic Inulin | 470.00 | mg |
| Sodium Bicarbonate (USP) | 10.00 | mg |
| Citric Acid (USP fine) | 5.00 | mg |
| Silicon Dioxide | 6.00 | mg |
| Organic Oat Fiber | 160.00 | mg |
| Organic Milk Thistle (whole seed) | 270.00 | mg |
| Silicon Dioxide | 17.00 | mg |

NONI

| Tablet Core Ingredients | Mass/Tablet | |
|---|---|---|
| Organic Inulin | 400.0 | mg |
| Sodium Bicarbonate (USP) | 16.0 | mg |
| Citric Acid (USP fine) | 8.0 | mg |
| Organic Safflower Oil | 25.0 | mg |
| Silicon Dioxide | 4.5 | mg |
| Noni Organic (NG) | 250.0 | mg |

VALERIAN

| | Mass/Tablet | |
|---|---|---|
| Tablet Core Ingredients | | |
| Organic Soy Fiber | 25 | mg |
| Organic Inulin | 300 | mg |
| Sodium Bicarbonate (USP) | 10.00 | mg |
| Citric Acid (USP fine) | 5.00 | mg |
| Organic Safflower Oil | 12 | mg |
| Silicon Dioxide | 5 | mg |
| Organic Oat Fiber | 25 | mg |
| Organic Valerian | 400.00 | mg |
| Tablet Coating Ingredients | | |
| Carageenan | 7.43 | mg |
| Glycerin USP Nat. | 0.39 | mg |

SOY

| Tablet Core Ingredients | Mass/Tablet | |
|---|---|---|
| Organic Inulin | 200.00 | mg |
| Sodium Bicarbonate (USP) | 10.00 | mg |
| Citric Acid (USP fine) | 5.00 | mg |
| Silicon Dioxide | 2.00 | mg |
| Organic Soy | 250.00 | mg |

SAW PALMETTO

| | Mass/Tablet | |
|---|---|---|
| Tablet Core Ingredients | | |
| Organic Inulin | 430.00 | mg |
| Sodium Bicarbonate (USP) | 7.00 | mg |
| Citric Acid (USP fine) | 4.00 | mg |
| Silicon Dioxide | 20.00 | mg |
| Organic Saw Palmetto Berries | 280.00 | mg |
| Tablet Coating Ingredients | | |
| Organic Honey | 6.55 | mg |
| Carageenan | 0.41 | mg |
| Glycerin USP Nat. | 0.28 | mg |
| Organic Soy Lecithin | 0.17 | mg |

ALOE JUICE

| Tablet Core Ingredients | Mass/Tablet | |
|---|---|---|
| Organic Inulin | 181.50 | mg |
| Sodium Bicarbonate (USP) | 9.50 | mg |
| Citric Acid (USP fine) | 6.00 | mg |
| Organic Safflower Oil | 18.00 | mg |
| Silicon Dioxide | 5.00 | mg |
| Organic Oat Fiber | 95.00 | mg |
| Organic Aloe Juice | 200.00 | mg |

BLACK COHOSH

| Tablet Core Ingredients | Mass/Tablet |
|---|---|
| Organic Inulin | 300.00 mg |
| Organic Safflower Oil | 27.00 mg |
| Silicon Dioxide | 13.00 mg |
| Organic Black Cohosh | 540.00 mg |

CALCIUM

| | Mass/Tablet | |
|---|---|---|
| Tablet Core Ingredients | | |
| Organic Inulin | 300.00 | mg |
| Organic Safflower Oil | 70.00 | mg |
| Silicon Dioxide | 9.60 | mg |
| Organic Gum Arabic | 30.58 | mg |
| Organic Eggshell | 810.81 | mg |
| Tablet Coating Ingredients | | |
| Organic Honey | 10.77 | mg |
| Carageenan | 0.70 | mg |
| Glycerin USP Nat. | 0.45 | mg |
| Organic Soy Lecithin | 0.29 | mg |

CRANBERRY

| | Mass/Tablet | |
|---|---|---|
| Tablet Core Ingredients | | |
| Organic Soy Fiber | 49.50 | mg |
| Organic Inulin | 57.00 | mg |
| Organic Safflower Oil | 5.50 | mg |
| Silicon Dioxide | 6.00 | mg |
| Organic Gum Arabic | 11.00 | mg |
| Organic Cranberry | 500.00 | mg |
| Tablet Coating Ingredients | | |
| Organic Honey | 5.55 | mg |
| Carageenan | 0.35 | mg |
| Glycerin USP Nat. | 0.24 | mg |
| Organic Soy Lecithin | 0.15 | mg |

ECHINACEA

| Tablet Core Ingredients | Mass/Tablet |
|---|---|
| Organic Inulin | 175.00 mg |
| Organic Safflower Oil | 18.00 mg |
| Silicon Dioxide | 15.00 mg |
| Organic Oat Fiber | 70.00 mg |
| Organic Echinacea (Angustifolia root) | 350.00 mg |

ECHINACEA-GOLDEN

| | Mass/Tablet | |
|---|---|---|
| Tablet Core Ingredients | | |
| Organic Inulin | 155.00 | mg |
| Organic Safflower Oil | 16.00 | mg |
| Silicon Dioxide | 50.00 | mg |
| Organic Cherry Fruit 10:1 | 13.00 | mg |
| Organic Oat Fiber | 61.00 | mg |
| Organic Echinacea-Golden (Angustifolia root) | 256.00 | mg |
| Tablet Coating Ingredients | | |
| Organic Honey | 4.86 | mg |
| Carageenan | 0.30 | mg |
| Glycerin USP Nat. | 0.21 | mg |
| Organic Soy Lecithin | 0.13 | mg |
| Purified Water USP | 0.00 | mg |
| Coating amount 1% | 555.5 | mg |

ORGANIC GARLIC SLOW

| | Mass/Tablet | |
|---|---|---|
| Tablet Core Ingredients | | |
| Organic Inulin | 780.50 | mg |
| Organic Safflower Oil | 45.00 | mg |
| Silicon Dioxide | 5.00 | mg |
| Organic Garlic | 302.50 | mg |
| Tablet Coating Ingredients | | |
| Organic Locust Bean | 50.00 | mg |
| Organic Guar | 55.00 | mg |
| Sodium Alginate | 59.00 | mg |

GARLIC

| Tablet Core Ingredients | Mass/Tablet | |
|---|---|---|
| Organic Inulin | 250.0 | mg |
| Sodium Bicarbonate (USP) | 17.00 | mg |
| Citric Acid (USP fine) | 12.00 | mg |
| Organic Safflower Oil | 40.0 | mg |
| Silicon Dioxide | 14.00 | mg |
| Organic Oat Fiber | 182.0 | mg |
| Organic Garlic | 605 | mg |

GINKGO

| | Mass/Tablet | |
|---|---|---|
| Tablet Core Ingredients | | |
| Organic Inulin | 350.00 | mg |
| Sodium Bicarbonate USP | 7.00 | mg |
| Citric Acid (USP fine) | 5.00 | mg |
| Organic Safflower Oil | 40.00 | mg |
| Silicon Dioxide | 8.00 | mg |
| Organic Ginkgo | 250.00 | mg |
| Tablet Coating Ingredients | | |
| Organic Honey | 5.83 | mg |
| Carageenan | 0.36 | mg |
| Glycerin USP Nat. | 0.25 | mg |
| Organic Soy Lecithin | 0.16 | mg |

GINSENG

| | Mass/Tablet | |
|---|---|---|
| Tablet Core Ingredients | | |
| Organic Inulin | 348.00 | mg |
| Sodium Bicarbonate USP | 7.00 | mg |
| Citric Acid (USP fine) | 5.00 | mg |
| Organic Safflower Oil | 35.00 | mg |
| Silicon Dioxide | 15.00 | mg |
| Organic Ginseng | 500.00 | mg |
| Tablet Coating Ingredients | | |
| Organic Honey | 8.04 | mg |
| Carageenan | 0.50 | mg |
| Glycerin USP Nat. | 0.34 | mg |
| Organic Soy Lecithin | 0.21 | mg |

MACA

| Tablet Core Ingredients | Mass/Tablet |
|---|---|
| Organic Inulin | 350.00 mg |
| Sodium Bicarbonate USP | 11.00 mg |
| Citric Acid (USP fine) | 9.00 mg |
| Organic Safflower Oil | 20.00 mg |
| Silicon Dioxide | 16.00 mg |
| Organic Gum Arabic | 5.00 mg |
| Organic Maca | 500 mg |
| | Granulate 50% of |
| | maca |

ALTERNATIVE MILK THISTLE

| Tablet Core Ingredients | Mass/Tablet | |
|---|---|---|
| Organic Inulin | 318.00 | mg |
| Sodium Bicarbonate USP | 10.50 | mg |
| Citric Acid (USP fine) | 7.50 | mg |
| Silicon Dioxide | 8.50 | mg |
| Organic Oat Fiber | 37.00 | mg |
| Organic Tapioca Dextrose | 6.50 | mg |
| Organic Milk Thistle (whole seed) | 280.00 | mg |

ST. JOHN'S WORT

| | Mass/Tablet | |
|---|---|---|
| Tablet Core Ingredients | | |
| Organic Inulin | 330.0 | mg |
| Sodium Bicarbonate USP | 12.0 | mg |
| Citric Acid (USP fine) | 9.0 | mg |
| Organic Safflower Oil | 30.0 | mg |
| Silicon Dioxide | 8.0 | mg |
| St. John's Wort | 300.0 | mg |
| Tablet Coating Ingredients | | |
| Organic Honey | 5.61 | mg |
| Carageenan | 0.382 | mg |
| Glycerin USP Nat. | 0.223 | mg |
| Organic Soy Lecithin | 0.159 | mg |

VITAMIN B12

| | Mass/Tablet | |
|---|---|---|
| Tablet Core Ingredients | | |
| Organic Inulin | 380.0 | mg |
| Organic Safflower Oil | 40.0 | mg |
| Silicon Dioxide | 8.0 | mg |
| Organic Oat Fiber | 140.0 | mg |
| Cyanocobalamin | 25.00 | mg |
| Organic Spirulina | 150.0 | mg |
| Tablet Coating Ingredients | | |
| Organic Honey | 6.57 | mg |
| Carageenan | 0.41 | mg |
| Glycerin USP Nat. | 0.28 | mg |
| Organic Soy Lecithin | 0.18 | mg |

Suitable tablet cores for the above examples may be prepared as generally discussed above. Preparation of all-natural tablet coating compositions and application thereof to tablet cores is described in commonly assigned, copending U.S. Provisional Application No. 61/026,315 (Attorney Docket No. ENZ01 PP-301), which is hereby incorporated by reference.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. An all-natural or substantially all-natural dietary supplement in a tablet form, comprising:
at least 95 percent Certified Organic ingredients by weight, the Certified Organic ingredients including at least one Certified Organic dietary supplement, and at least a first tablet excipient selected from naturally occurring substances, the first tablet excipient being present in an amount effective to act as a tablet binder, a tablet disintegrant, or both a tablet binder and a tablet disintegrant.

2. A dietary supplement as in claim 1, wherein the substance is a dietary fiber.

3. A dietary supplement as in claim 1, wherein the substance is oat fiber.

4. A dietary supplement as in claim 1, wherein the substance is soy fiber.

5. A dietary supplement as in claim 1, wherein the substance is dextrose.

6. A dietary supplement as in claim 1, wherein the substance is inulin fiber.

7. A dietary supplement as in claim 1, further comprising a second tablet excipient that is a Certified Organic ingredient selected from monosaccharides, disaccharides, oligosaccharides, and polysaccharides, the second tablet excipient being present in an amount effective to act either alone or in combination with the first tablet excipient as a tablet binder, a tablet disintegrant, or both a tablet binder and a tablet disintegrant.

8. A dietary supplement as in claim 7, wherein the first and second tablet excipients are inulin and soy fiber.

9. A dietary supplement as in claim 7, wherein the first and second tablet excipients are inulin and oat fiber.

10. A dietary supplement as in claim 7, wherein the first and second tablet excipients are inulin and dextrose.

11. A dietary supplement as in claim 7, wherein the first and second tablet excipients are dextrose and oat fiber.

12. A dietary supplement as in claim 7, wherein the first excipient, the second excipient, and a third excipient are inulin, soy fiber and oat fiber.

13. A dietary supplement as in claim 1, further comprising an effervescent combination.

14. A dietary supplement as in claim 1, further comprising a tablet lubricant, the tablet lubricant being a Certified Organic oil disposed on surfaces of a Certified Organic polysaccharide.

15. A dietary supplement as in claim 1, further comprising a tablet lubricant, the tablet lubricant comprising a Certified Organic oil disposed on surfaces of a Certified Organic maltodextrin.

16. A dietary supplement as in claim 1, further comprising a tablet lubricant, the tablet lubricant comprising a Certified Organic oil disposed on surfaces of a Certified Organic gum acacia.

17. A dietary supplement in a compressed tablet form, comprising:
from 5 percent to 80 percent by weight of a combination of at least two tablet excipients selected from the group consisting of Certified Organic carbohydrates, at least one of the tablet excipients being a dietary fiber, the combination of tablet excipients acting as a tablet binder, a tablet disintegrant, or both a tablet binder and a tablet disintegrant; and
a nutritionally or botanically effective amount of a Certified Organic dietary supplement comprising up to about 95 percent by weight of the tablet;
from about 0.1 percent to about 3 percent by weight of a tablet lubricant comprising Certified Organic oil disposed on surfaces of a Certified Organic polysaccharide;
the dietary supplement in a compressed tablet form comprising from 95 percent to 100 percent Certified Organic ingredients by weight, and from 0 to 5% by weight of organic compliant ingredients.

18. A dietary supplement as in claim 17, wherein the tablet lubricant comprises the Certified Organic oil disposed on surfaces of a Certified Organic maltodextrin.

19. A dietary supplement as in claim 17, wherein the tablet lubricant comprises the Certified Organic oil disposed on surfaces of a Certified Organic gum acacia.

20. A dietary supplement as in claim 17, wherein the tablet excipients include a combination of inulin and Certified Organic soy fiber.

21. A dietary supplement as in claim 17, wherein the tablet excipients include a combination of inulin and Certified Organic oat fiber.

22. A dietary supplement as in claim 17, wherein the tablet excipients include a combination of inulin and dextrose.

23. A dietary supplement as in claim 17, wherein the tablet excipients include a combination of Certified Organic oat fiber and Certified Organic soy fiber.

24. A dietary supplement as in claim 17, further comprising an effervescent combination.

25. A process for making a dietary supplement in a tablet form, comprising:
providing a selected Certified Organic dietary supplement in a nutritionally effective amount;
optionally granulating the dietary supplement with a Certified Organic ingredient that acts as a granulating agent to form a granulated dietary supplement;
providing at least one tablet excipient selected from naturally occurring substances, the tablet excipient being present in an amount effective to act as a tablet binder, a tablet disintegrant, or both a tablet binder and a tablet disintegrant;
blending the dietary supplement or granulated dietary supplement with the at least one tablet excipient, and, optionally, with other ingredients to provide a tablet formulation containing at least 95 percent Certified Organic ingredients by weight; and
compressing the tablet formulation to form a tablet.
